(19) United States
(12) Patent Application Publication
(10) Pub. No.: US 2024/0412847 A1
(43) Pub. Date: Dec. 12, 2024

(54) AI TCM DIAGNOSTIC AND ADVICE SYSTEM

(71) Applicant: Maishen Inc., Santa Clara, CA (US)

(72) Inventors: Dalong Zhang, Santa Clara, CA (US); Xinghe Chen, Santa Clara, CA (US)

(73) Assignee: Maishen Inc., Santa Clara, CA (US)

(21) Appl. No.: 18/659,392

(22) Filed: May 9, 2024

(51) Int. Cl. G16H 20/90; G16H 10/20

(52) U.S. Cl. CPC G16H 20/90; G16H 10/20

(57) ABSTRACT

An online Traditional Chinese Medicine (TCM) diagnostic system allows users to recognize possible health conditions according to TCM's theory of 12 Meridians. When a user enters their phone number, a unique divination technique is applied to a unique method of listing the 12 Meridian organs, the program asks the user a set of questions for confirmation. Depending on the user's response, the invention generates a health information report and makes TCM product recommendations.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 is a block diagram of a computing environment for an online diagnostic system that allows users to interact with the AI Diagnostic Robot, according to one embodiment.

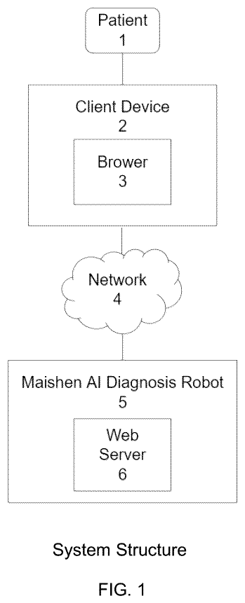

FIG. 2 is a block diagram of an online diagnostic system that shows the interaction between patient information, host management, and development environment, according to one embodiment.

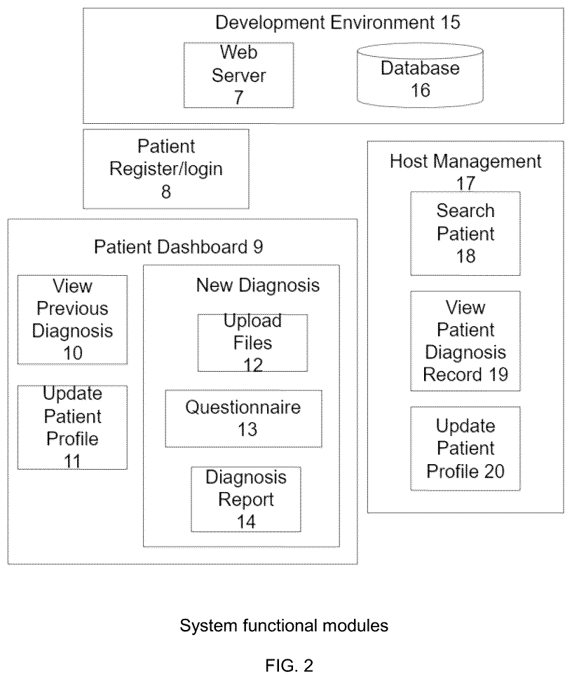

FIG. 3 is a flowchart of user input to generate a diagnostic report, according to one embodiment.

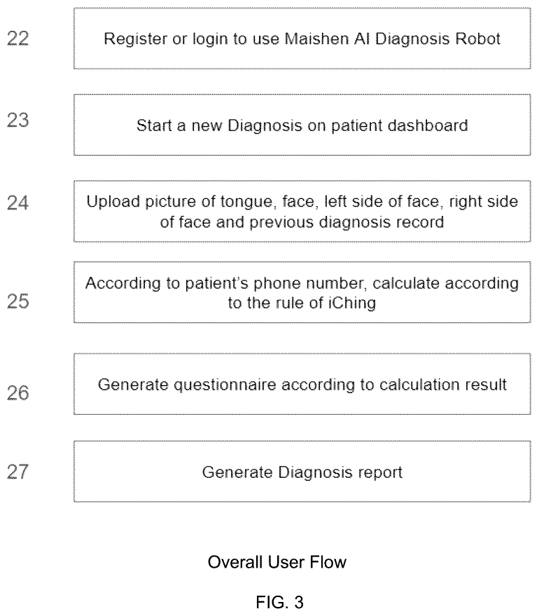

FIG. 4 is a flowchart of login or registration of user, according to one embodiment.

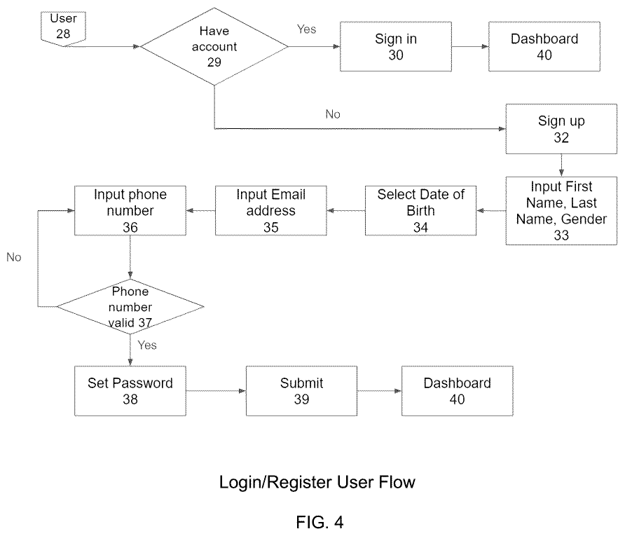

FIG. 5 is a flowchart of diagnostic of user, according to one embodiment.

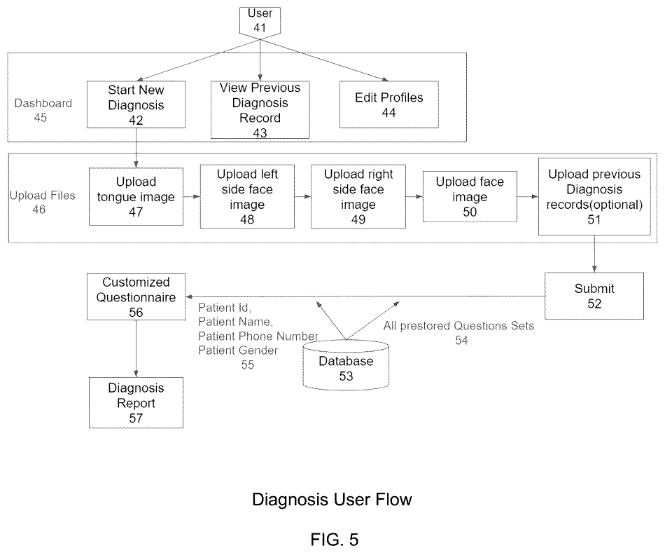

FIG. 6 is a flowchart of the lung questionnaire, according to one embodiment.

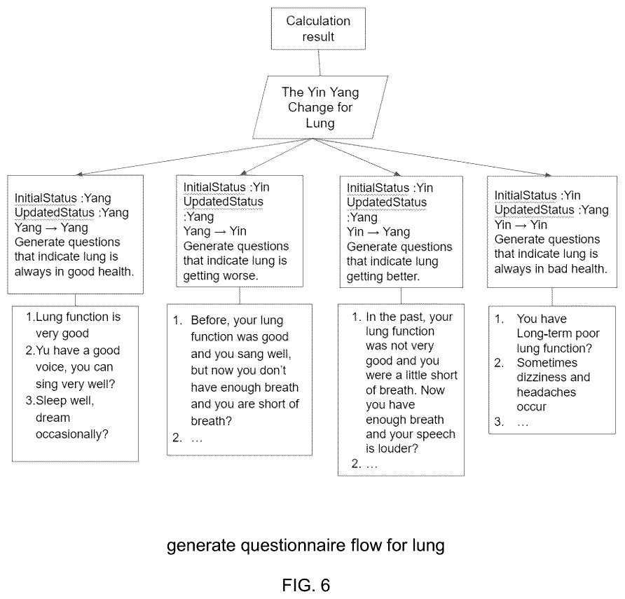

FIG. 7 is a flowchart of the pericardium questionnaire, according to one embodiment.

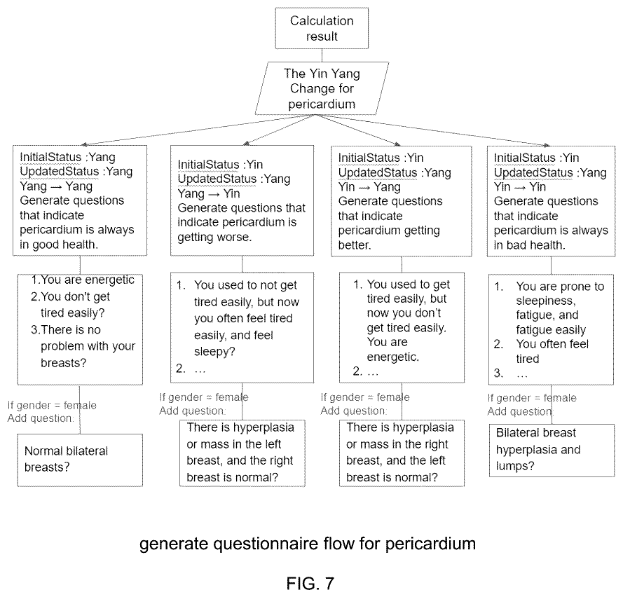

FIG. 8 is a flowchart of the heart questionnaire, according to one embodiment.

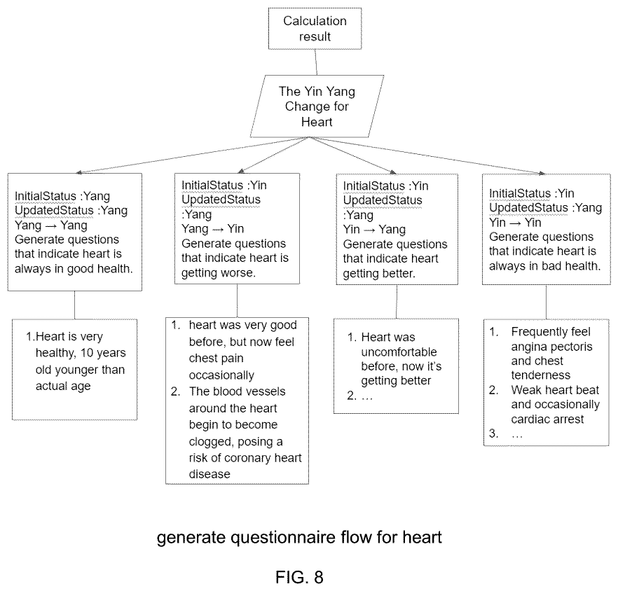

FIG. 9 is a flowchart of the stomach questionnaire, according to one embodiment.

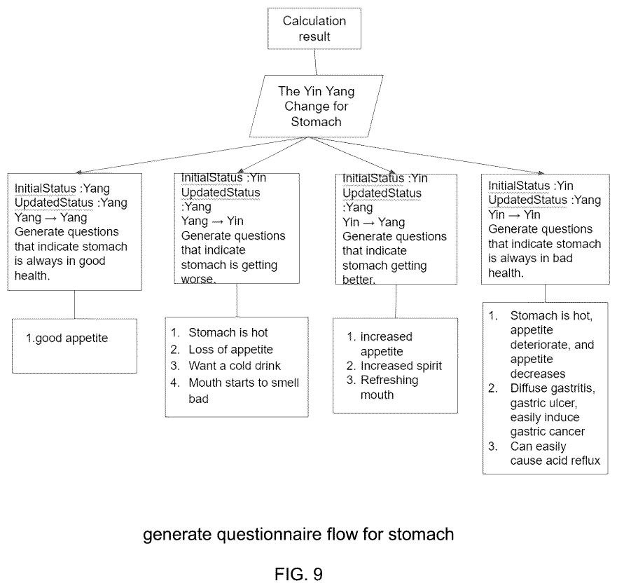

FIG. 10 is a flowchart of the gallbladder questionnaire, according to one embodiment.

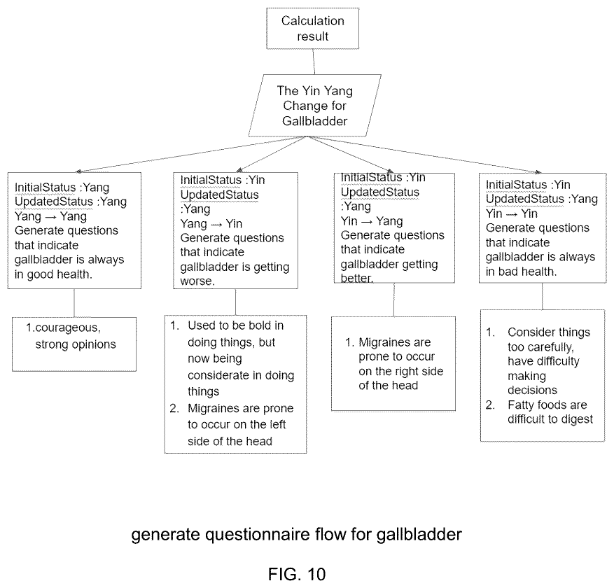

FIG. 11 is a flowchart of the urinary bladder questionnaire, according to one embodiment.

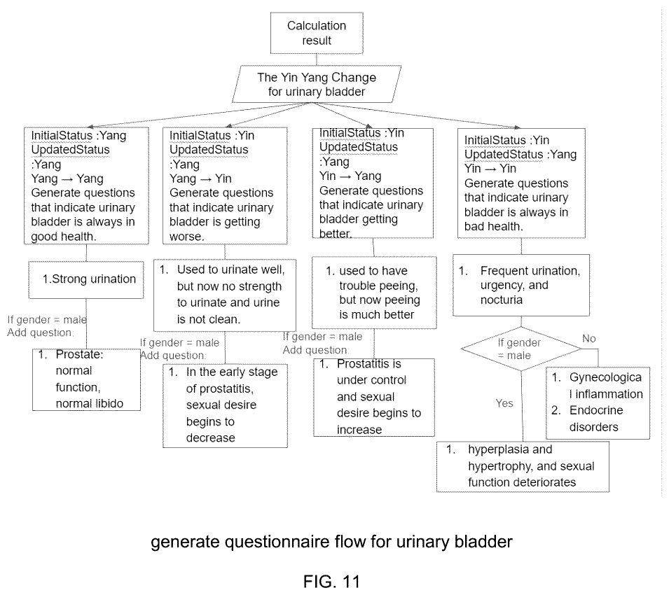

FIG. 12 is a flowchart of the spleen questionnaire, according to one embodiment.

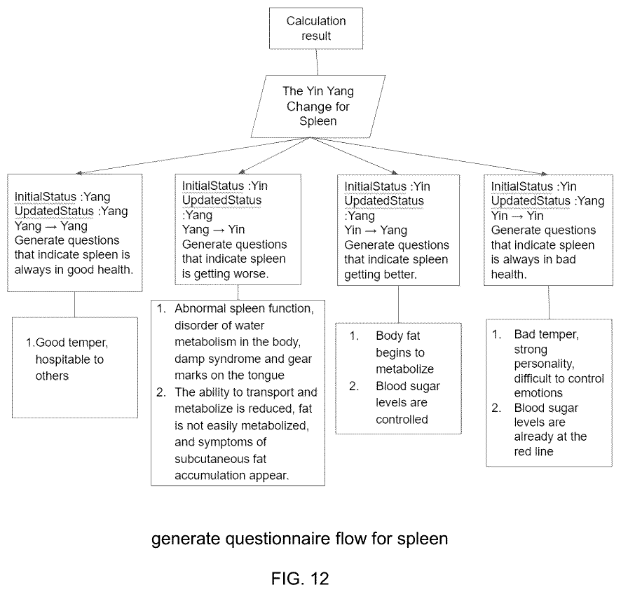

FIG. 13 is a flowchart of the liver questionnaire, according to one embodiment.

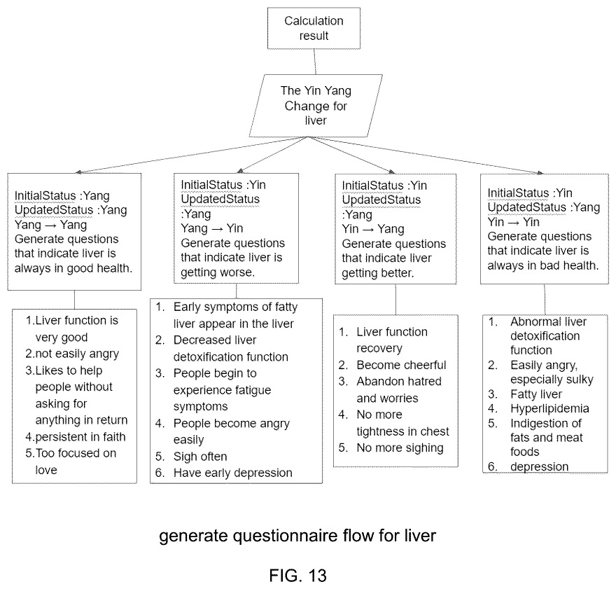

FIG. 14 is a flowchart of the kidney questionnaire, according to one embodiment.

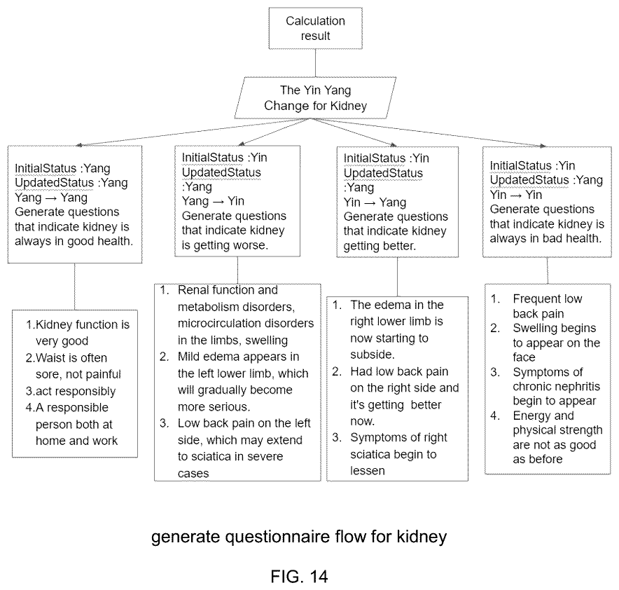

FIG. 15 is a flowchart of the large intestine questionnaire, according to one embodiment.

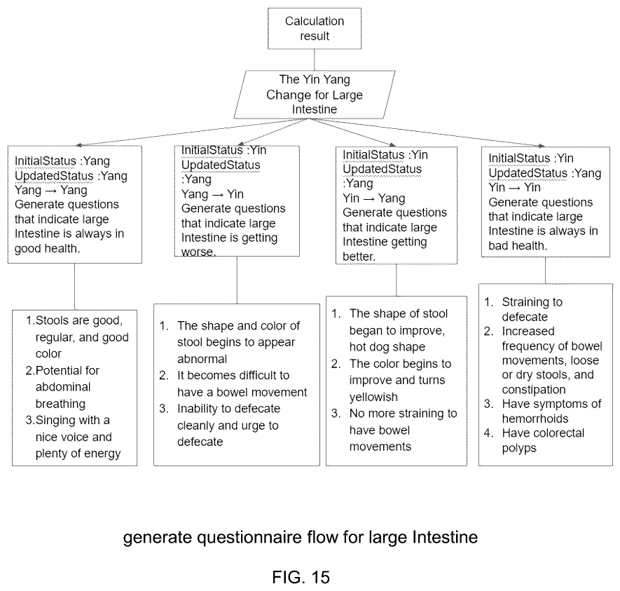

FIG. 16 is a flowchart of the triple burner questionnaire, according to one embodiment.

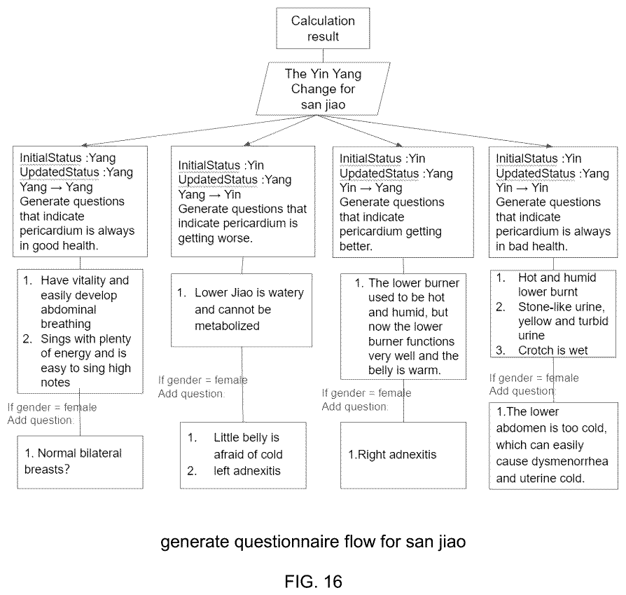

FIG. 17 is a flowchart of the small intestine questionnaire, according to one embodiment.

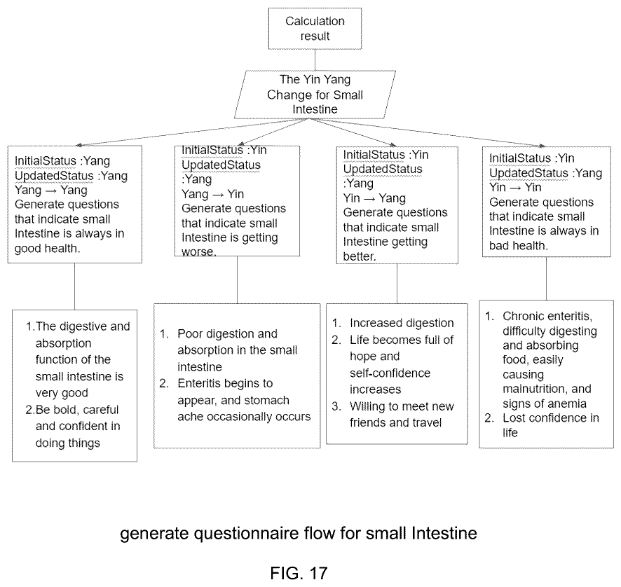

FIG. 18 is the order of placement of the last three non-zero phone number digits with organs, according to one embodiment.

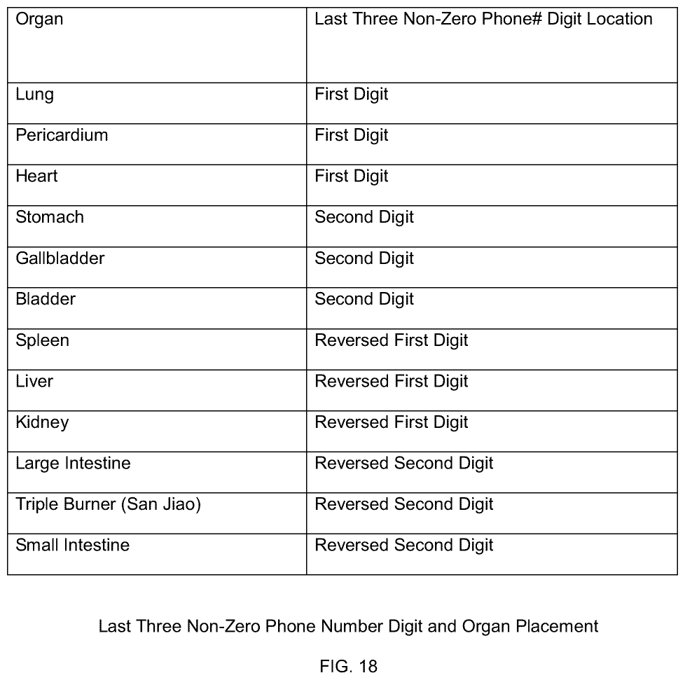

FIG. 19 is a picture showing TCM fingertip-to-wrist positions, Cun, Guan, Chi.

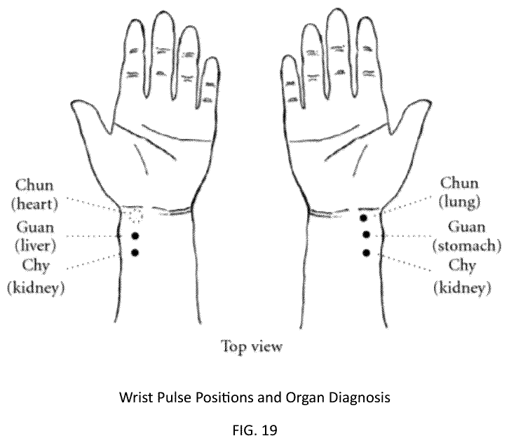

FIG. 20 is a diagram depicting IChing Trigrams and its corresponding number for calculation.

### SUMMARY OF THE INVENTION

An online TCM diagnostic system allows users to implement an ancient Chinese divination technique to recognize possible conditions of the 12 organs according to the TCM theory of 12 Meridians. When a user enters their phone number, the numbers are calculated with a unique divination method, applying the results to a unique listing of each internal organ of the 12 Meridians, and based on the results of this method, the program asks the user a set of questions. Depending on these methods and the user's response to the questionnaire, the invention generates a health informational report and makes product recommendations.

### BACKGROUND

A recent survey shows Four in 10 Americans put off going to the doctors and 52% say the cost of healthcare is the primary reason. Indeed, healthcare is expensive. An average American spends about $13,500 per year on healthcare. With so many Americans who forgo regular health checkups because of cost, a solution is needed to encourage them to learn about possible health implications as cheaply as possible.

Other than cost, the reasons surveyors say they avoid receiving regular health checkups include anxiety about the test or exhaustion from parenting or caretaking. Anxiety from the diagnostic test results stem from being in a medical setting, surrounded by intrusive technologies that might be intimidating to patients who are protective of their physical boundaries.

With so many Americans unable to afford regular health checkups and anxious about diagnostic results, it is important to encourage them to be informed about possible implications to their health, doing so cheaply, and to provide health information to them at their own convenience.

More than 2,500 years ago, Traditional Chinese Medicine discovered Acupuncture, Pulse Diagnosis and the Meridian System as a method to treat various illnesses including pain relief, reducing symptoms of cold and flu. The National Institute of Health finds that acupuncture for pain relief tends to have the most evidence, especially for conditions that have become chronic like osteoarthritis and lower back pain, as well as tension headaches.

Just as there are channels in our body to transport blood and oxygen, and a networked system to send signals in the central nervous system, TCM believes there are different channels in the body to transport blood and Qi (vital energy). In TCM, Jing means meridian or sometimes channels of energy, and Luo means ‘net’, sometimes translated as ‘collaterals.’ Together, Jing Luo is believed to be channels in the body that are connected to internal organs and acupuncture points. Generally, when the Qi and blood within the Meridians are balanced and unobstructed, it is indicative of good health.

According to TCM, the movement of Qi and blood creates a pulse, with its circulation maintained by the involvement of heart, lung and liver Qi. The spleen is believed to produce Qi and is stored in the kidneys. Thus, all internal organs play a role in producing a pulse and maintaining a healthy circulation in the body.

To detect whether the internal organs are healthy, and Qi and blood are unobstructed, TCM practitioners measure the pulse by placing their fingertips on the patients' inner wrist. The finger tips are placed in three positions: distal, proximally joint-wrist space (close to the styloid process of the radius), and proximal. The wrist positions in which pulse diagnosis is taken is referred to as Cun, Guan, Chi, respectively. TCM practitioners apply three different pressures on the patients' wrist (light, medium, and firm), to feel pulse palpitations. Each wrist-position is connected to a Meridian or an organ, and by feeling the pulse palpitations, TCM practitioners can diagnose the organ conditions. TCM practitioners consider the depth (superficial or deep), rate (slow or rapid), shape (frequency and amplitude), flow (forceful or weak), and rhythm (regular or irregular), to make a complete picture. It is widely-known that this technique takes a tremendous amount of study and time to master.

In TCM, there are 12 primary Meridians and 8 extraordinary meridians, with accompanying and connecting structures that are located at different depths. The 12 meridian channels, each connecting to several major organs are, the Lung meridian, Large Intestine meridian, Stomach meridian, Spleen meridian, Heart meridian, Small Intestine meridian, Bladder meridian, Kidney meridian, Pericardium meridian, Triple Burner meridian, Gall Bladder meridian, and Liver meridian. There are a total of 361 points located on the meridian, each point is applied with different techniques to balance and tonify qi, including acupuncture, moxibustion, micro massage, Chinese massage, electrostimulation, laser, and electrical nerve stimulation.

### IChing

To the Chinese, the IChing is equivalent to the Holy Bible of the West. The most honored sages in history, Fu Xi, King Wen, the Duke of Zhou, contributed to different parts of the text. Confucius' commentaries, composed of ten chapters, is often referred to as the Ten Wings, for they believed the IChing depends on the Ten Wings to fly.

The IChing cannot be easily understood without Confucius' commentaries.

IChing was initially a handbook for divination but after Confucius commentary, it became a book of insight and wisdom. For those who consult with the IChing, they learn about the possibilities of the present situation, future potential and instructions for what to do and what not to do to obtain fortune and avoid misfortune.

This iconic book reveals ancient wisdom, the Tao (way) of change. For centuries, the Chinese observed the stars, constellations, growth, and decay, and the five elements. They believe the symbols represents certain truths about our relationship with nature and civilization. They found that humans, the progeny of the cosmos in integrated form, share a universal principle within all things, change. Thus, the IChing is sometimes referred to as the Book of Changes. These symbolic concepts, propagated by the IChing, have been the source of Chinese culture and thinking for thousands of years.

The imageries written in the IChing have gone through a few transformations of its own. The book was known as the I of the Xia dynasty (2005-1766 B.C.) and the I of the Shang dynasty (1766-1122 B.C.) then the I of the Zhou dynasty (1122-221 B.C.) although only fragments of the two earlier books survived.

Fu Xi, from the Xia dynasty, originated the eight primary trigrams. King Wen of Zhou arranged each trigram on top of another, forming the sixty-four hexagrams and wrote the decisions for each. King Wen's son, Duke of Zhou, composed the Yao Texts. Later, in the Zhou dynasty, Confucius wrote the Ten Wings commentary. In the Han dynasty (206 B.C. to A.D. 220) a new edition of the IChing appeared with five of Confucius' ten commentaries printed beside the original text. This version of the IChing became the most revered edition and played a central role in Chinese thinking for two thousand years.

A trigram (gua) is formed by three horizontal lines, stacked on top of another. Each line (yao) can be broken (yin), or complete (yang). When two trigrams, the lower trigram and the upper trigram, are arranged on top of another, a hexagram of eight lines is formed. The lower trigram is sometimes referred to as the inner gua, the upper trigram is sometimes referred to as the outer gua. Each line of the hexagram can also be a changing from broken (yin) line to complete (yang) line, or from yang (complete) line to broken (yin) line, each changing line was provided an interpretation in the original text.

Each hexagram, composed of 384 possible changing lines, over 500 items of judgment or decisions are distributed amongst them. Every line has at least one judgment of insight, good fortune or misfortune.

The attributes of yin (broken lines) and yang (complete lines) are exact opposites that complements. Yang lines symbolize the Heavens, masculine, the firm, the strong, to advance, the odd numbers, as well as all active things. Yin lines symbolize the Earth, feminine, the yielding, the receptive, to retreat, the even numbers, as well as all passive things. Traditionally, the six lines are arranged from the bottom to the top, just as the bottom is the foundation for all things to grow upward.

In the same way Chinese medicine believes there are five elements constituting physical functions, each trigram also represents the five elements, properties that exists in all things. These five elements are wood, fire, earth, metal, and water. In addition to the five elements each trigram represents, they also symbolize elements of nature: heaven, lake, fire, thunder, wind, water, mountain and earth. When heaven is stacked on top of earth, the image and symbol is peace. When reversed, the image and symbol is stagnation, for heaven is apart from earth, the creative is distant from the receptive, leading to confusion and disorder.

The traditional method to consult the IChing is through the use of yarrow stalks, fifty of them, manipulated three times to produce one line. For a hexagram, fifty yarrow stalks need to be manipulated eighteen times. At least half an hour is required to get a hexagram. Two thousand years later, in the Tang dynasty (618-907) a simpler way of using three coins was introduced. Sometimes the changing lines are derived from variations of the same method but historically, yarrow stalks and three coins were the most common methods used.

The purpose of divination was to resolve doubts and confusion. Ancient historic records show that the practice of divination far predated the time of IChing. The court of the Shang dynasty (1766-1122 B.C.) divined with tortoise shells, to consult about grave matters, such as war expeditions, enemy invasions, weather disasters, seasonal ceremonies, government administrations, weddings, and hunts.

### DETAILED DESCRIPTION

The present invention does not provide advice for every Traditional Chinese Medicine (TCM) problem, nor does it make an exhaustive study of one vertical cross-section of medicine. Instead, the present invention provides information about possible health conditions for twelve internal organs according to the teachings of TOM.

In one embodiment there is a computerized method of organ diagnosis according to TOM and IChing divination, the method comprising ascertaining a plurality of attributes representative of an organ condition of a patient, wherein an attribute is associated with the phone number of a user.

In another embodiment there is a computerized method of organ diagnosis according to TCM and IChing divination, the method comprising ascertaining a plurality of attributes representative of an organ condition of a patient, wherein an attribute is associated with the order of listing of organs for the placement IChing divination.

In another embodiment there is a computerized method of organ diagnosis according to TCM and IChing divination, the method comprising ascertaining a plurality of attributes representative of an organ condition of a patient, wherein a first attribute is associated with the phone number of a user and a second attribute is associated with a TCM product suggestion.

In another embodiment there is a computerized method of organ diagnosis according to TCM and IChing divination, the method comprising ascertaining a plurality of attributes representative of an organ condition of a patient, wherein a first attribute is associated with the phone number of a user and a second attribute is associated with a questionnaire for confirmation of IChing calculations.

In another embodiment there is a computerized method of organ diagnosis according to TCM and IChing divination, the method comprising ascertaining a plurality of attributes representative of an organ condition of a patient, wherein a first attribute is associated with the phone number of a user and a second attribute is associated with gender of a user, and a third attribute is associated with a questionnaire for confirmation of IChing calculations.

In another embodiment there is a computerized method of organ diagnosis according to TCM and IChing divination, the method comprising ascertaining a plurality of attributes representative of an organ condition of a patient, wherein a first attribute is associated with the phone number of a user and a second attribute is associated with gender of a user, and a third attribute is associated with a questionnaire for confirmation of IChing calculations, and a fourth attribute is associated with TCM product recommendations.

For convenience, the following description will be outlined into the following: Introduction, systems overview, input module, calculation module, response module, and output module.

### Introduction

One of the drawbacks of the traditional TCM doctor-patient relationship is the short amount of time that TCM practitioners are able to spend with patients. The present invention, however, allows patients as much time as they wish to learn about their problem as well as to obtain information on other medical topics.

The most serious drawback of pulse diagnosis in TCM is that practitioners are only able to detect 7 organs, with 6 fingertip positions placed on the patient's wrist, and some positions indicate two organs, further complicating this problem. The three positions TCM practitioners' fingertips are placed on is referred to as Cun, Guan, Chi. The fingertip-to-wrist positions are the same for both the right and left hand, for a total of six fingertip-to-wrist positions. The pulse conditions of the left hand Cun, Guan, Chi positions indicate heart, liver and gallbladder, left kidney, respectively. The pulse conditions of the right hand Cun, Guan, Chi positions indicate lung, spleen and stomach, and right kidney, respectively.

When TCM practitioners diagnose a patient's pulse, they are only able to detect a few of the 12 Meridian organs. Since many of the 12 Meridians do not reach the wrist, TCM practitioners are often required to use other TCM methods for a complete diagnosis. Further, the pulse diagnostic results of left-hand Guan position, and results of the right-hand Guan position covers two organs each, which needs to be further verified with other TCM methods, I.e. tongue coating diagnostic.

| Position | Left Hand | Right Hand |
|---|---|---|
| Cun | Heat | Lung |
| Guan | Liver and Gallbladder | Stomach and Spleen |
| Chi | Kidney | Kidney |

Using a unique method of IChing technique with the user's phone number, practitioners can diagnose the conditions of the 12 organs according to the theory of 12 Meridians. When a user enters their phone number, the numbers are calculated with a unique divination method, applying the results to a unique listing of each internal organ of the 12 Meridians, and based on the results of this method, the system makes product recommendations.

In addition, the program asks the user a set of leading affirmative/negative questions for confirmation. In at least one instance, If the user responses no to all questions, diagnostic reports will not be shown, but the health condition percentages will be shown. If the user responds yes to any question, both a diagnostic report and a health condition percentage will be shown. Depending on these methods and the user's response to the questionnaire 8, the invention generates a health informational report 14.

### Systems Overview

The present inventions use standard industry components, and offer portability for other operating systems. While specific hardware and software will be referenced, it will be understood that a panoply of different components could be used in the present system.

In Chinese medicine, practitioners spend valuable time duplicating tasks such as pulse diagnosis, asking questions related to the conditions of the 12 Meridians. The present invention utilizes TCM knowledge from thousands of years of research, to diagnose the most commonly encountered problems in Chinese Medicine. These algorithms are carefully and specifically designed to diagnose 12 Meridian conditions from different sets of questionnaires and pulse diagnosis results if available.

Several TCM practitioners can work together to thoroughly apply pulse diagnostics as well as to anticipate the possible problems and patient responses. These experts could also provide and maintain the present invention's product/herbal treatment recommendation table.

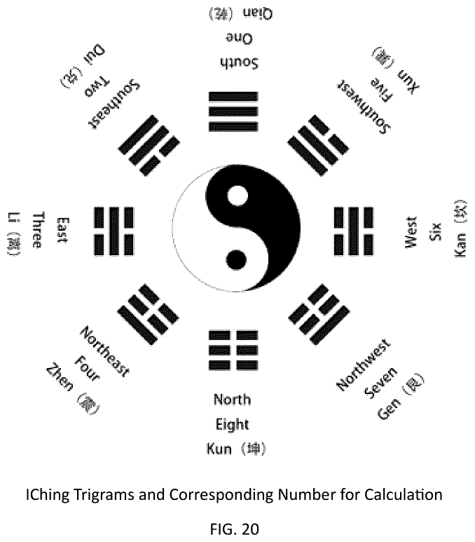

This present invention determines the user's organ condition based on a unique calculation technique applying ancient Chinese divination methods. The calculation module takes the last three non-zero digits of the user's phone number to obtain first set G1: G1(FirstDigit), G1(SecondDigit), and G1(ThirdDigit); and reverses the last three-non-zero digit to obtain the second set: G2(FirstDigit), G2(SecondDigit), and G2(ThirdDigit). If the first or second set's first two digits are larger than 8, then 8 is subtracted from that digit. If the first or second set's last digit is greater than 6, then 6 is subtracted from that digit. From the result of this calculation, we obtain the first two digits, each digit represents a trigram from FIG. 20. The first-digit trigram is stacked on top of the second-digit trigram. From this hexagram we compare these complete and broken lines from top to bottom, to the following order (from top to bottom): Lung, Pericardium, Heart, Stomach, Gallbladder, Bladder, Spleen, Liver, Kidney, Large Intestine, Triple Buner, and Small Intestine.

Here, as an example, Qian, with three complete lines, corresponds with the number one. Kun, with three broken lines, corresponds with the number eight.

When stacking the first set's two digit on top of another, a hexagram (8 lines) is formed. If the G1(FirstDigit) is 3 and the G1(SecondDigit) is 2, 3 will be stacked on top of 2, and the resulting hexagram will have the following features from top to bottom: broken (yin), complete (yang), broken (yin), broken (yin), complete (yang), complete (yang).

The last digits of both sets G1(ThirdDigit) and G2(ThirdDigit) will represent the changing line, counted from the bottom up. Using the example above, if the ThirdDigit is a 4, the fourth line from the bottom up is a broken (yin) line changed to a complete (yang) line. This indicates that the health condition of the organ is changing from poor condition to good condition.

These lines are drawn next to a list of organs with the following order (from top to bottom): Lung, Pericardium, Heart, Stomach, Gallbladder, Bladder, Spleen, Liver, Kidney, Large Intestine, Triple Burner, and Small Intestine. If the line next to the organ is broken (yin), the diagnostic indicates the organ is in poor condition. If the line next to the organ is complete (yang), the calculation indicates the organ is in good condition. If the line is changing from broken (yin) to complete (yang), the calculation indicates the organ is transforming from poor to good condition. If the line is changing from complete (yang) to broken (yin), the calculation indicates the organ is transforming from good to poor condition.

| | Last 3 Digits: 556, 655. | Changing | |
|---|---|---|---|
| Organ | Yin/Yang | Line? | Condition |
| Lung | Yang | Yes | Good to Poor |
| Pericardium | Yang | No | Good |
| Heart | Yin | No | Poor |
| Stomach | Yang | No | Good |
| Gallbladder | Yang | No | Good |
| Bladder | Yin | No | Poor |
| Spleen | Yin | No | Poor |
| Liver | Yang | Yes | Good to Poor |
| Kidney | Yin | No | Poor |
| Large Intestine | Yang | No | Good |
| Triple Burner | yang | No | Good |
| Small Intestine | Yin | No | Poor |

Carefully crafted questions, used in the taking of a pulse diagnosis, are the main tools that the present invention uses to assess the problems of customers. The key to getting a good result is to ask the right questions. In a sense, in the diagnostic process questions are like tests. Although they may be refined over time, in general, once excellent and well-crafted questions are developed, they can be used for a long time. Of course, as new research uncovers knowledge about TCM, new sets of diagnostic questions are developed that address this specific concern.

The questions used by an earlier generation of TCM practitioners, who did not have the latest research in TCM, are far more unreliable and imprecise in diagnosing a patient's problem than the questions used by TCM practitioners today. The present invention makes use of fine nuances of language to diagnose patients as well as to confirm whether the calculations are accurate.

For the present invention, when the calculation results show that the user's organ is in good condition, the system will not recommend a product to the user. But if the calculation results show that the user's organ is in poor condition, the system will recommend a Fuheng herbal product to the user.

Different TCM treatment products are offered depending on the diagnostic results. If the diagnostic is conclusive, and the organ is in poor condition, different products are offered to treat the 12 organs. The products offered if the diagnostic results are as follows:

| Organ | Product Recommendation |
|---|---|
| Lung | Fuheng Lung Tea |
| Pericardium | Fuheng Heart Tea |
| Heart | Fuheng Enhance Organ Function |
| Stomach | Fuheng Stomach Tea |
| Gallbladder | Fuheng Wen Dan Tea |
| Urinary Bladder | Fuheng Frequent Urination Tea |
| Spleen | Fuheng Qu Shi Tea |
| Liver | Fuheng Liver Tea |
| Kidney | Fuheng Kidney Tea |
| Large Intestine | Fuheng Laxative Tea |
| San Jiao | Fuheng Detox Tea |
| Small Intestine | Fuheng Digestive Tea |

The present invention's statistical generating capabilities enable the system to confirm the accuracy of the IChing calculations used in the diagnostic process. Depending on the result of IChing calculation, a different set of questionnaires are used to confirm calculations, either affirmative leading questions or negative leading questions. If the result from the questionnaire shows the user has responded no to all questions, the calculations are inconclusive, and only the health percentages will be shown and the diagnostic results will not show. For example, if a customer's lung is diagnosed in poor condition from the calculations, the questionnaire will ask affirmative leading questions such as “Do you easily run out of breath?” If the user answers no to all of the negative leading questions, then the health percentages will be shown and the diagnostic results will not show. Conversely, if a customer's lung is diagnosed in good condition from the calculations, the questionnaire will ask negative leading questions such as “You don't easily run out of breath?” If the user answers no to all of the negative leading questions, the IChing calculation is inconclusive. As such, only the health percentages will be shown and the diagnostic result will not be shown. With this method, TCM practitioners benefit from the immense amount of statistical information that is gathered regarding the wording of questions asked in taking symptoms related to the 12 Meridians.

In addition, for different organs, the systems will add additional questions for varying genders and with one additional organ, the system will report a different diagnostic result. For female Pericardium and Triple Burner, additional questions are added for confirmation. For male bladder, additional questions are added for confirmation. Lastly, for bladder, different diagnostic reports are shown for male or females.

### Input Module

A software module that allows users to enter their mobile phone number and collect the user's phone number during the registration process. Upon receiving the mobile phone number, the verification module triggers an SMS gateway to send a unique, time-sensitive verification code to the specified number. The verification code expires shortly after it is sent to ensure that it cannot be reused by unauthorized users. The input module verifies the code entered by the user against the sent code to confirm authenticity. A secure storage system holds registered phone numbers along with verification statuses and user details. Communications between the mobile phone and the verification server are encrypted.

The system will ask the user to upload a picture of tongue, front face, left side of the face, right side of the face and previous diagnostic record.

The module can be implemented on various platforms such as web, mobile, and desktop applications, ensuring broad accessibility and utility. For example, an online banking application uses this system to verify new users during account setup, providing an additional layer of security by confirming the ownership of the mobile phone number listed in the registration form.

Communication between the user's device and the server, including the transmission of the phone number and verification code, is encrypted to prevent unauthorized access.

Technical specifications include software and hardware that are necessary to implement and support the system, such as specific server, database, and SMS gateway integrations.

### Calculation Module

The present invention asks questions used to identify problems related to the 12 Meridians and 12 organs.

In another embodiment, the system asks the patient for some information in different ways at different times, then compares the patient's responses to determine if they are consistent. If not, the system automatically omits responses entered by the user.

In the present invention, modularity and modifiability are important. Modularity is the key to the system's ability to provide customers with the most current medical information available. The invention's system design and techniques allow the individual components of the system to be easily modified. Once the medical algorithms are written and programmed, they can then be continuously updated and refined as advances in TCM are made. The ability to nearly instantaneously modify the present invention is a unique feature of the system.

The calculation module of the present invention first determines the user's health condition based on calculations of the last three non-zero digits of the user's phone number, where the last three non-zero digits of the user's phone number are refined to two sets of three-digit numbers.

The module then scans the mobile phone number from right to left to identify and extract Set three variables: firstDigit, secondDigit and thirdDigit to store the last three non-zero digits of a user's mobile phone number and identify them as the first group of numbers.First Group (G1): Consists of the last three non-zero digits extracted from the phone number. Second Group (G2): Formed by reversing the digits of the first group. For example, the phone number is 1+345-560-0078, then the first group of numbers is 678, and the second group of numbers is 876.

Transformation Rule (R1) is as follows:

Transformation for First Two Digits: For each of the first two digits in both groups, if the digit is greater than 8, for example, it is 9, the module subtracts 8 from it. That digit changed from 9 to 1.

Transformation for the Last Digit: For the last digit in each group, if the digit is greater than 6, for example, it is 8, the module subtracts 6 from it. The last digit changed from 8 to 2.

### Here is the Processing Logic Example 1:

If the first group is ‘987’, after applying the transformation, the numbers are adjusted as follows:
First digit: 9 (greater than 8) becomes 1 (9-8)
Second digit: 8 (equal to 8) remains 8
Third digit: 7 (greater than 6) becomes 1 (7-6).
Resulting in the transformed group: ‘181’

Similarly, the second group (formed by reversing ‘987’ to ‘789’) will be transformed accordingly.
First digit: 7 (smaller than 8) remains 7
Second digit: 8 (equal to 8) remains 8
Third digit: 9 (greater than 6) becomes 3 (9-6)
Resulting in the transformed group: ‘783’
Here is the processing logic example 2:
If the phone number 1-875-374-208 is first group is ‘428’, after applying the transformation, the numbers are adjusted as follows:
   First digit: 4 (smaller than 8) remains 4
   Second digit: 2 (smaller to 8) remains 2
   Third digit: 8 (greater than 6) becomes 2 (8-6)
   Resulting in the transformed group: ‘422’
Similarly, the second group (formed by reversing ‘428’ to ‘824’) will be transformed accordingly.
   First digit: 8 (equal to 8) remains 8
   Second digit: 2 (smaller than 8) remains 2
   Third digit: 4 (smaller than 6) remains 4
   Resulting in the transformed group: ‘783’
Every digit of the first two digits for each group of numbers represents three changes of Yin and Ying. The rule is as follows:
   1: Yang Yang Yang.
   2: Yin Yang Yang.
   3. Yang Yin Yang.
   4. Yin Yin Yang.
   5. Yang Yang Yin.
   6. Ying Yang Yin.
   7. Yang Yin Yin.
   8. Yin Yin Yin

### Organ-Yin-Yang Association:

First Set of Organs (S1): Lung, Pericardium, and Heart, Stomach, Gallbladder, and Bladder.
Second Set of Organs (S2): Spleen, Liver, and Kidney, Large Intestine, Triple Warmer, and Small Intestine.
   Each digit in the first and second sets is associated with a specific organ and its yin-yang balance. The analysis process is structured as follows:
      First Set (S1) organs Associations:
         First digit: Determines the yin-yang status of the Lung, Pericardium, and Heart.
         Second digit: Determines the yin-yang status of the Stomach, Gallbladder, and Bladder.
         Third digit: Count the value backwards, thereby changing the yin and yang to one of the organs in the first Set (S1), changing it from yang to yin, or from yin to yang. And the others in the second set of the organs remain the same Yin or Yang.
      Second Set (S2) organs Associations:
         First digit: Determines the yin-yang status of the Spleen, Liver, and Kidney.
         Second digit: Determines the yin-yang status of the Large Intestine, Triple Warmer, and Small Intestine.
         Third digit: Count the value backwards, thereby changing the yin and yang to one of the organs in the first Set (S2), changing it from yang to yin, or from yin to yang. And the others in the second set of the organs remain the same Yin or Yang.

Each organ's health condition is assessed based on the transition of states: A transition from Yin to Yin indicates continued ill health. A transition from Yin to Yang indicates improvement from ill health to health. A transition from Yang to Yin indicates a decline from health to ill health. A transition from Yang to Yang indicates sustained health.
Algorithm Implementation.
   The module gets the user's phone number, scans from left to right, and finds three non-zero digits of the user's phone number. And conduct the transformation rule (R1), it gets transformed two groups of numbers G1, G2
   Setup two sets of organs S1 (Lung, Pericardium, and Heart, Stomach, Gallbladder, and Bladder), S2 (Spleen, Liver, and Kidney, Large Intestine, Triple Warmer, and Small Intestine).
   According to the first digit of the first group G1, we can get 8 kinds of yin and yang conditions for lungs, pericardium, and heart. We set it as a diagram (D1). D1 has 8 kinds of Yin or Yang conditions for three organs. That is the First Digit (possibly 1,2,3,4,5,6,7,8) of S1 associated with G1. And in our algorithm, we use False to represent Yang, True represents Ying. So we get the following 8 situations, this is D1:
      1: lung: False, pericardium: False, heart: False
      2: lung: True, pericardium: False, heart: False
      3: lung: False, pericardium: True, heart: False
      4: lung: True, pericardium: True, heart: False
      5: lung: False, pericardium: False, heart: True
      6: lung: True, pericardium: False, heart: True
      7: lung: False, pericardium: True, heart: True
      8: lung: True, pericardium: True, heart: True
   According to the second number, we can get 8 possible yin and yang conditions for stomach, gallbladder, and bladder. For the yin and yang situation, we set it as a diagram 2 (D2).
      1: stomach: False, gallbladder: False, urinaryBladder: False
      2: stomach: True, gallbladder: False, urinaryBladder: False
      3: stomach: False, gallbladder: True, urinaryBladder: False
      4: stomach: True, gallbladder: True, urinaryBladder: False
      5: stomach: False, gallbladder: False, urinaryBladder: True
      6: stomach: True, gallbladder: False, urinaryBladder: True
      7: stomach: False, gallbladder: True, urinaryBladder: True
      8: stomach: True, gallbladder: True, urinaryBladder: True.
   According to the first digit of the second group, we can get the yin and yang of the spleen, liver, and kidney. There may be 8 yin and yang situations. We set it as a diagram 3 (D3).
      1: spleen: False, liver: False, kidney: False
      2: spleen: True, liver: False, kidney: False
      3: spleen: False, liver: True, kidney: False
      4: spleen: True, liver: True, kidney: False
      5: spleen: False, liver: False, kidney: True
      6: spleen: True, liver: False, kidney: True
      7: spleen: False, liver: True, kidney: True
      8: spleen: True, liver: True, kidney: True
   According to the first digit of the second group, we can get the yin and yang of the spleen, liver, and kidney. The second digit in the second group determines the yin and yang conditions of the large intestine, triple burner, and small intestine. There may be 8 yin and yang conditions. We set it as a diagram 4 (D4)
      1: large intestine: False, san jiao: False, small intestine: False
      2: large intestine: True, san jiao: False, small intestine: False
      3: large intestine: False, san jiao: True, small intestine: False.
      4: large intestine: True, san jiao: True, small intestine: False.
      5: large intestine: False, san jiao: False, small intestine: True.
      6: large intestine: True, san jiao: False, small intestine: True
      7: large intestine: False, san jiao: True, small intestine: True.
      8: large intestine: True, san jiao: True, small intestine: True
According to D1, D2, D3, D4, this module inputs the first two digits of G1 and G2, we can find the Initial Yin or Yang status for S1 and S2. It is called InitialStatus.
Next, this module counts the value of the third digit backwards, thereby changing the yin and yang of the organ in both S1 and S2. Change it from yang to yin, or from yin to yang. And the others in both sets of the organs remain the same Yin or Yang. The updated Yin or Yang for organs S1 and s2 is called UpdatedStatus.

| | | | | | | Show organ | |
|---|---|---|---|---|---|---|---|
| Organ | | Yin/Yang | Changing | Questionnaire | Product | health | Diagnostic |
| Condition | Set # | Line | Line? | Conclusive? | recommendation | percentage? | Report? |
| Lung | G1 | Yin | No | Yes | Fuheng | Yes | Yes |
| | (FirstDigit) | | | | Lung Tea | | |
| Pericardium | G1 | Yang | No | No | Fuheng | Yes | No |
| | (FirstDigit) | | | | Heart Tea | | |
| Heart | G1 | Yang | No | Yes | None | Yes | Yes |
| | (FirstDigit) | | | | | | |
| Stomach | G1 | Yin | Yes | Yes | None | Yes | Yes |
| | (SecondDigit) | | | | | | |
| Gallbladder | G1 | Yang | No | Yes | None | Yes | Yes |
| | (SecondDigit) | | | | | | |
| Bladder | G1 | Yang | Yes | Yes | Frequent | Yes | Yes |
| | (SecondDigit) | | | | Urination Tea | | |
| Spleen | G2 | Yin | Yes | No | None | Yes | No |
| | (FirstDigit) | | | | | | |
| Liver | G2 | Yang | No | No | Fuheng | Yes | No |
| | (FirstDigit) | | | | Liver Tea | | |
| Kidney | G2 | Yang | No | Yes | None | Yes | Yes |
| | (FirstDigit) | | | | | | |
| Large | G2 | Yang | No | No | Fuheng | Yes | No |
| Intestine | (SecondDigit) | | | | Laxative Tea | | |
| Triple | G2 | Yin | Yes | Yes | None | Yes | Yes |
| Burner | (SecondDigit) | | | | | | |
| Small | G2 | Yin | No | Yes | Fuheng | Yes | Yes |
| Intestine | (SecondDigit) | | | | Digestive Tea | | |

### Response Module

The present invention generates a different set of questions to ask the user based on results from the calculation module. These questions serve to confirm calculation results. Each organ has 4 sets of questions, to confirm the organ is in good condition (yang), poor condition (yin), or moving from good condition (yang) to poor condition (yin), or moving from poor condition (yin) to good condition (yang). QS1 is a set of questions that confirms this organ is maintaining good health. QS2 is a set of questions that confirms this organ is maintaining poor health. QS3 is a set of questions that confirms this organ is transitioning from good health to poor health. QS4 is a set of questions that confirms this organ is transitioning from poor health to good health.

For each organ, if the InitialStatus and UpdatedStatus are the same, so both of them are False (Yang), then this module selects Question set (QS1) to display. If the InitialStatus and UpdatedStatus are the same, both of them are True (Ying), then this module selects Question set (QS2) to display. If the InitialStatus and UpdatedStatus are NOT the same, InitialStatus is False (Yang), UpdatedStatus is True (Ylng), then this module selects Question set QS3 to display. If the InitialStatus and UpdatedStatus are NOT the same, InitialStatus is True (Ylng), UpdatedStatus is False (Yang), then this module selects Question set QS4 to display.

For three organs, pericardium, triple burner and bladder, when the gender of the patient is female, more specific questions are added to confirm diagnosis for women. When the gender of the patient is male, add more specific questions for men.

### Output Module

Under certain conditions, the Output Module produces a diagnosis report for each organ. When calculation results show that the user's organ is in good condition, the system will not recommend a product to the user. But if the calculation results show that the user's organ is in poor condition, the system will recommend a Fuheng herbal product to the user.

The present invention's statistical generating capabilities enable the system to confirm the accuracy of the IChing calculations used in the diagnostic process. Depending on the result of IChing calculation, a different set of questionnaires are used to confirm calculations, either affirmative leading questions or negative leading questions. If the result from the questionnaire shows the user has responded no to all questions, the calculations are inconclusive, and only the health percentages will be shown and the diagnostic results will not show.

For example, if a customer's lung is diagnosed in poor condition from the calculations, the questionnaire will ask affirmative leading questions such as “You easily run out of breath?” If the user answers no to all of the negative leading questions, then the health percentages will be shown and the diagnostic results will not show. Conversely, if a customer's lung is diagnosed in good condition from the calculations, the questionnaire will ask negative leading questions such as “You don't easily run out of breath?” If the user answers no to all of the negative leading questions, the IChing calculation is inconclusive. As such, only the health percentages will be shown and the diagnostic result will not be shown. With this method, TCM practitioners benefit from the immense amount of statistical information that is gathered regarding the wording of questions asked in taking symptoms related to the 12 Meridians.

Different TCM treatment products are offered depending on the diagnostic results. If the diagnostic is conclusive, and the organ is in poor condition, different products are offered to treat the 12 organs. The products offered if the diagnostic results are as follows:

| Organ | Product Recommendation |
|---|---|
| Lung | Fuheng Lung Tea |
| Pericardium | Fuheng Heart Tea |
| Heart | Fuheng Enhance Organ Function |
| Stomach | Fuheng Stomach Tea |
| Gallbladder | Fuheng Wen Dan Tea |
| Urinary Bladder | Fuheng Frequent Urination Tea |
| Spleen | Fuheng Qu Shi Tea |
| Liver | Fuheng Liver Tea |
| Kidney | Fuheng Kidney Tea |
| Large Intestine | Fuheng Laxative Tea |
| San Jiao | Fuheng Detox Tea |
| Small Intestine | Fuheng Digestive Tea |

In addition, for different organs, the systems will add additional questions for varying genders and with one additional organ, the system will report a different diagnostic result. For female Pericardium and Triple Burner, additional questions are added for confirmation. For male bladder, additional questions are added for confirmation. Lastly, for bladder, different diagnostic reports are shown for male or females.

### Additional Considerations

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments of the invention may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

1. A computer implemented method, comprising:
Receiving a phone number from a client device;
Computing, by a computer processor, the last three non-zero digits of a phone number, to obtain the first set;
Re-computing, by a computer processor, the digits within the first set, if the first two digits are greater than eight then subtract eight from that digit, and if the last digit is greater than six then subtract six from that digit;
Computing, by a computer processor, the last three non-zero digits of a phone number, reversing the last three non-zero digits of a phone number, to obtain the second set;
Re-computing, by a computer processor, the digits within the second set, if the first two digits are greater than eight then subtract eight from that digit, and if the last digit is greater than six then subtract six from that digit;
Listing, by a computer processor, the first set of organs by the order of first to last, or top to bottom, Lung, Pericardium, Heart, Stomach, Gallbladder, Bladder;
Listing, by a computer processor, the second set of organs by the order of first to last, or top to bottom, Spleen, Liver, Kidney, Large Intestine, Triple Burner, Small Intestine;
Associating, by a computer processor, the first two digits of the first set and the second set with yin or yang, according to traditional Chinese Bagua;
Associating, by a computer processor, the last digit of the first set and the last digit of the second set, with the changing of yin to yang, or yang to yin;
Reporting, by a computer processor, organ results based on yin or yang, where yin indicates poor organ condition, yang indicates good organ condition, yin changing to yang indicates poor organ condition changing to good organ condition, and yang changing to yin indicates good organ condition changing to poor organ condition;

2. The method of claim 1, wherein varying sets of questionnaires are asked to confirm the calculation of organ conditions either in the form of leading affirmative questions, or leading negative questions.

3. The method of claim 1, wherein additional sets of questionnaires are asked to confirm the calculations of organ conditions depending on the gender of the user.

4. The method of claim 1, wherein if the calculation results in poor organ condition, a TCM product is recommended.

5. The method of claim 1, wherein an organ condition, from the calculations, is shown in a pie chart, showing percentages of an organ in good condition and poor conditions.

6. The method of claim 1, wherein a diagnostic report is shown to explain the organ conditions as a result of the calculations.

7. The method of claim 1, wherein the system will ask the user to upload a picture of the user's tongue, front face, left side of the face, right side of the face, and previous diagnostic record.

8. A computer implemented method, comprising:
Receiving a diagnostic report from a client device, the diagnostic report including a plurality of conditions;
Computing, by a computer processor, sets of questionnaires to present to the user depending on the conditions from the diagnostic report;
Reporting, by a computer processor, the health conditions of the user.

9. The method of claim 8, wherein varying sets of questionnaires are asked to confirm the calculation of health conditions either in the form of leading affirmative questions, or leading negative questions.

10. The method of claim 8, wherein additional sets of questionnaires are asked to confirm the calculations of health conditions depending on the gender of the user.

11. The method of claim 8, wherein if the calculation results in poor organ condition, a TCM product is recommended.

12. The method of claim 8, wherein an organ condition, from the calculations, is shown in a pie chart, showing percentages of health conditions.

13. The method of claim 8, wherein a diagnostic report is shown to explain the health condition as a result of the calculations.

14. The method of claim 8, wherein the system will ask the user to upload a picture of the user's tongue, front face, left side of the face, right side of the face, and previous diagnostic record.
